(19) United States
(12) Patent Application Publication
(10) Pub. No.: US 2024/0342351 A1
(43) Pub. Date: Oct. 17, 2024

(54) MULTI-PART DIALYZER

(71) Applicant: B. Braun Avitum AG, Melsungen (DE)

(72) Inventor: Peter Mandry, Dresden (DE)

(21) Appl. No.: 18/683,040

(22) PCT Filed: Aug. 10, 2022
(86) PCT No.: PCT/EP2022/072426

§ 371 Date: Feb. 12, 2024

(30) Foreign Application Priority Data
Aug. 13, 2021 (DE) 10 2021 121 108.5

(51) Int. Cl. A61M 1/16

(52) U.S. Cl. CPC A61M 1/1621; A61M 1/168

(57) ABSTRACT

A housing-free first dialyzer element has a hollow-fiber bundle, a fluid-permeable sheath wrapped around the periphery of the hollow-fiber bundle, cast elements at the axial ends of the hollow-fiber bundle, and end caps fastened to the cast elements for connection to an extracorporeal blood circulation. A second dialyzer element has a housing and can be closed, opened and reused. A dialyzer includes the housing-free first dialyzer element and the second dialyzer element that has a housing. A blood treatment device includes the second dialyzer element that has the housing, the second dialyzer element being rigidly fastened to a machine front. A method for reprocessing the second dialyzer element that has the housing can be used after blood treatment therapy.

### CROSS-REFERENCE TO RELATED APPLICATIONS

This application is the United States national stage entry of International Application No. PCT/EP2022/072426, filed on Aug. 10, 2022, which claims priority to German Application 10 2021 121 108.5, filed on Aug. 13, 2021. The contents of International Application No. PCT/EP2022/072426 and German Application 10 2021 121 108.5 are incorporated by reference herein in their entireties.

### FIELD

The present disclosure relates to a multi-part dialyzer. In particular, it relates to a first dialyzer element or respectively dialyzer part without housing, a second dialyzer element or respectively dialyzer part with housing, a dialyzer comprising the first dialyzer element without housing and the second dialyzer element with housing, a blood treatment device comprising a machine front and the second dialyzer element with housing, and a method for reprocessing the second dialyzer element with housing after a blood treatment therapy.

### BACKGROUND

A dialyzer basically has a plurality of hollow fibers that form a hollow fiber bundle. The hollow fiber bundle is cast or embedded in a casting compound in a known manner (see e.g. EP 0 844 015 B1). Together with the casting compound, the hollow fiber bundle is inserted into a cylindrical (dialyzer) housing during assembly of the dialyzer and is glued into this housing, for example using polyurethane. Finally, the hollow fiber bundle is closed via two caps at the axial ends of the dialyzer.

In this context, it is known from DE 198 06 293 A1, for example, to wrap the hollow fiber bundle with a sheathing film made of paper or plastic during the manufacture or production of the dialyzer. The sheathing film holds the hollow fibers together and, in particular, protects the outer hollow fibers from damage when the hollow fiber bundle is inserted into the housing. After the hollow fiber bundle has been inserted into the housing, the sheathing film is usually removed again, since it would impede a dialysis fluid flow or dialysate flow and thus a uniform flow around the hollow fibers when the dialyzer is used as intended in the context of blood treatment therapy.

The cylindrical (dialyzer) housing into which the hollow fiber bundle is inserted is usually a one-piece (hollow) cylindrical housing part. However, a two-part housing is also known from EP 1 466 657 B1, which—if the hollow fiber bundle is inlaid—is closed in an unopenable manner by welding or gluing.

After blood treatment therapy/dialysis, all parts contacting the blood and thus also the entire dialyzer are usually disposed of and thus destroyed. Normally, only dialysis fluid-carrying/dialysate-carrying parts are disinfected and reused. This results in large quantities of plastic waste. Against this background, it would generally be desirable to reduce the amount of plastic waste that has to be disposed of after blood treatment therapy or to avoid plastic waste as far as possible.

It is already known from the state of the art, for example from CN 201044830 Y or EP 0 864 334 B1, to reprocess dialyzers after their utilization/use as part of a blood treatment therapy. However, the reprocessing of an entire dialyzer has proven to be time-consuming and labor-intensive and entails microbiological and hygienic risks and problems.

### SUMMARY

Against this background, the object of the present disclosure is to avoid or at least reduce the disadvantages of the prior art. In particular, sustainability for a dialysis is to be improved by reducing plastic waste (keyword: green dialysis). Furthermore, a dialysis patient should not be endangered with a possibly still contaminated, reprocessed dialyzer.

Advantageous embodiments are explained below.

The present disclosure is based on the core idea of forming or constructing a dialyzer with multiple parts, in particular in two parts. In particular, according to the disclosure, the blood-carrying or blood-contacting parts/portions/regions of the dialyzer are combined in a first component, i.e. the first dialyzer element or respectively dialyzer part without housing according to the disclosure, or are formed by the first component, and the dialysis fluid-carrying/dialysate-carrying or respectively dialysis fluid-contacting/dialysate-contacting parts/portions/regions of the dialyzer are combined in a second component, namely the second dialyzer element or respectively dialyzer part with housing according to the disclosure, or are formed by the second component. If the blood-carrying parts of the dialyzer are constructed separately from the dialysis fluid-carrying parts of the dialyzer, the dialysis fluid-carrying parts, i.e. the second dialyzer element with housing, can advantageously remain on the blood treatment device/dialysis machine after a blood treatment therapy and can be disinfected and/or cleaned, i.e. reprocessed for reuse, and the blood-carrying parts of the dialyzer, i.e. the first dialyzer element or respectively dialyzer part without housing, can be removed and disposed of.

In other words, according to the present disclosure, it has been realized that the housing of the dialyzer is usually disposed of together with the entire dialyzer after the blood treatment therapy, although it is not in contact with the blood, since it is inseparably connected to the blood-carrying parts of the dialyzer. Against this background, according to the present disclosure, the housing of the dialyzer (the second dialyzer element with housing) is separable from the blood-carrying parts (the first dialyzer element without housing). Thus, the plastic waste in the dialyzer can be greatly reduced.

The present disclosure relates firstly to a first dialyzer element/dialyzer part without housing comprising: a hollow fiber bundle comprising a first axial end and a second axial end (opposite the first axial end); a liquid-permeable/fluid-permeable sheathing/sheathing film/sleeve (circumferentially) wrapped around the hollow fiber bundle (and thus packaging the hollow fiber bundle); a first cast element/cast cap provided at the first axial end of the hollow fiber bundle and a second cast element/cast cap provided at the second axial end of the hollow fiber bundle; and a first end cap/blood cap attached to the first cast element and a second end cap/blood cap attached to the second cast element, wherein the first dialyzer element without housing, in particular the first end cap and the second end cap, is (are) prepared and provided to be connected to an extracorporeal blood circuit.

According to the disclosure, the first dialyzer element without housing does not have a housing. In other words, this is omitted in the manufacture of the first dialyzer element without housing. According to the present disclosure, a housing is understood to mean a solid sheathing that protects a (housing) content from environmental influences such as liquids. The sheathing/sheathing film/sleeve of the present disclosure is not to be regarded as a housing, since it is liquid-permeable and is also intended to allow liquids to pass through when the first dialyzer element without housing is used as intended. In this context, it should also be noted that the sheathing or sheathing film remains on the first dialyzer element without housing during the intended use of the first dialyzer element without housing in the context of a blood treatment therapy and is not removed again before the intended use, as in DE 198 06 293 A1.

This means that immediately before the intended utilization/use of the substantially cylindrical or rotationally symmetrical first dialyzer element without housing, the two end caps at the axial ends of the first dialyzer element without housing and the liquid-permeable sheathing represent the interface or interfaces of the first dialyzer element without housing to the environment.

The sheathing is preferably formed or configured in such a way that it allows dialysis fluid flow or dialysate flow through it. In other words, the sheathing preferably has a negligible resistance for dialysis fluid or dialysate, i.e. allows liquids such as dialysis fluid or dialysate in particular to pass through it substantially unhindered.

In addition, the sheathing is preferably wrapped around the hollow fiber bundle in such a way that the hollow fibers of the hollow fiber bundle are held together circumferentially, i.e. are secured circumferentially by the sheathing. This ensures or enables secure bundling of the hollow fibers and, in particular, the outer hollow fibers are suitably protected, e.g. during inserting the first dialyzer element without housing into a separate second dialyzer element with housing (which is separate from the first dialyzer element without housing).

In an advantageous manner, the sheathing is wrapped at least once, preferably twice, around the hollow fiber bundle. In other words, the sheathing/sheathing film has at least sectionally overlapping portions.

It is furthermore advantageous if the sheathing is made of a weldable material. This enables a secure closure of the sheathing without additional adhesive strips. For example, overlapping portions of the sheathing can be welded together.

A particularly preferred configuration example is characterized in that an inner end/an inner layer and an outer end/an outer layer of the sheathing overlap, wherein an intermediate layer of the sheathing is provided, so that at least sectionally three layers of the sheathing/sheathing film are superimposed. According to this configuration example, the two outer layers of the sheathing are preferably welded together, while the inner layer of the sheathing serves as a protective layer for the fibers.

The sheathing is preferably formed as a textile surface structure/textile structure, i.e. as a two-dimensional textile product. The sheathing is particularly preferably formed from a coarse-meshed fabric or a nonwoven and/or has a net-like structure.

The textile surface structure (for example the coarse-meshed fabric or the nonwoven or the net-like structure) is preferably configured or formed in such a way that it enables a dialysis fluid flow/dialysate flow through it and at the same time securely bundles the hollow fibers. Furthermore, it is preferably weldable. In other words, according to the disclosure, properties and materials of the textile surface structure, i.e. in particular of the coarse-mesh fabric/nonwoven/net-like structure, are suitably determined in order to fulfill the stated requirements. For example, a mesh size of a coarse-meshed fabric may be defined or selected in such a way that it has a negligible resistance for a dialysis fluid flow/dialysate flow. Meshes or openings of a net/net-like structure may also be set accordingly. In the case of a nonwoven, for example, the nonwoven reinforcement may be carried out in such a way that the resulting nonwoven fabric is liquid-permeable. In addition, a nonwoven may also be at least approximately net-like with a correspondingly large mesh size in order to provide the desired properties. One possible material for the textile surface structure is a weldable plastic material. The person skilled in the art will adjust or determine the properties or materials of the selected textile surface structure in order to provide the desired liquid permeability, secure bundling of the hollow fibers, and weldability in accordance with the present disclosure.

The nonwoven material disclosed in EP 3 017 100 B1, which is marketed by Low & Bonar B.V. under the registered trademark COLBACKR WA 30, has proven to be a particularly preferred textile surface structure for the sheathing.

The first cast element/the first cast cap and the second cast element/the second cast cap are preferably each produced by casting/molding the hollow fiber bundle at its first axial end and its second axial end via a casting compound and then cutting it. In particular, it is advantageous in this context if the textile surface structure of the sheathing also has a negligible resistance to the casting compound.

The casting compound is preferably a polyurethane or silicone compound, i.e. the cast elements/cast caps are preferably made of polyurethane or silicone. However, other materials that are suitable for preventing blood flow between the individual hollow fibers, i.e. in a clearance between the hollow fibers, are also possible. In particular, compared to a conventional dialyzer, other materials, for example biocompatible hardening liquids such as water glass, may be considered, since the cast elements do not need to form an adhesive bond with a (dialyzer) housing and therefore do not need to have any special adhesive properties.

In an advantageous manner, the first end cap is glued to the first cast element and/or the second end cap is glued to the second cast element in order to achieve a suitable attachment of the end caps to the cast elements.

Preferably, the first end cap comprises a first (tube) terminal and the second end cap comprises a second (tube) terminal, wherein the first terminal is provided to be connected to a first (blood-carrying) tube and the second terminal is provided to be connected to a second (blood-carrying) tube. Particularly preferably, the first dialyzer element without housing has exactly two terminals, i.e. the first terminal and the second terminal, so that the first dialyzer element without housing is prepared or configured to be connected to the extracorporeal blood circuit/blood-carrying tubes only/exclusively via the first terminal and the second terminal. In other words, the first dialyzer element without housing preferably has no dialysis fluid terminals/dialysate terminals, i.e. no terminals which are prepared, configured or provided to be connected to a dialysis fluid circuit/dialysate circuit.

Preferably, during a blood treatment therapy, extracorporeal blood of a patient enters the first dialyzer element without housing via the first terminal of the first end cap, flows through the hollow fibers, and leaves the first dialyzer element without housing via the second terminal of the second end cap. Thus, the first dialyzer element without housing is preferably a blood-carrying component/part/element of a dialyzer. In other words, blood preferably flows through the first dialyzer element without housing via the end caps and the hollow fibers, i.e. does not flow between the individual hollow fibers or through the sheathing.

Preferably, the end caps each have at least one (hollow) cylindrical/sleeve-shaped portion. The (hollow) cylindrical portion is preferably a portion with a constant inner diameter (over a length of the portion) and a constant outer diameter. The inner diameter of the (hollow) cylindrical portion corresponds particularly preferably substantially to a diameter of the substantially cylindrical cast element, so that the cast element can be inserted into the (hollow) cylindrical portion of the end cap in order to be attached, in particular glued, to an inner surface of the end cap.

The end caps may also each have a portion tapering towards the first terminal or the second terminal. Alternatively, however, a step-like transition between the (hollow) cylindrical portion and the first terminal or the second terminal may be provided. Other transitions are also conceivable.

The first terminal and the second terminal preferably extend in an axial direction of the first dialyzer element without housing, i.e. are aligned parallel to the hollow fiber bundle. However, the end caps may basically also be shaped in such a way that a blood supply and a blood discharge take place laterally, for example at a 90° angle.

According to the disclosure, the end caps do not have to be identically shaped, but should at least both be able to be attached to a cast element in a suitable manner and should preferably have a suitable terminal for a blood-carrying tube.

Preferably, the first end cap has a first sealing surface or a first sealing element on the outer circumference, i.e. on its outer diameter, and/or the second end cap has a second sealing surface or a second sealing element on the outer circumference, i.e. on its outer diameter.

In other words, according to the disclosure, two preferred embodiments are conceivable for the two end caps, i.e. for the first end cap and/or the second end cap, with regard to the sealing elements or sealing surfaces. According to a first embodiment, the end caps may have a sealing element, for example a sealing ring that is in particular molded or glued on, which is configured or provided to interact in a sealing manner with a sealing surface provided on a separate second dialyzer element with housing. Alternatively, that is, according to a second embodiment, the end caps may have a sealing surface which is configured or provided to interact in a sealing manner with a sealing element provided on a separate second dialyzer element with housing. According to the present disclosure, a sealing surface is to be understood as a surface which is basically suitable for interacting in a sealing manner with a sealing element, i.e. in particular a smooth, flat surface, preferably a cylindrical lateral surface if the sealing element is a sealing ring.

In principle, the first dialyzer element without housing may also be connected to a blood-carrying, exchangeable cassette, which in turn has terminals for blood coming from a patient and flowing back to the patient. According to this embodiment, the end caps or the terminals of the end caps are connected to the extracorporeal blood circuit via the blood-carrying, exchangeable cassette.

Preferably, the first dialyzer element without housing (without a solid housing) is packaged sterile. In other words, the first dialyzer element without housing consisting of the hollow fiber bundle, the sheathing, the two cast elements, and the two end caps is in sterile packaging. Prior to utilization/use of the first dialyzer element without housing, it is unpacked from the sterile packaging and is preferably inlaid/inserted into a separate second dialyzer element with housing described below.

The present disclosure thus further relates to a second dialyzer element with housing, which is formed as a closable, openable and reusable housing, comprising: a first housing part and a second housing part which are configured to be brought in an open state/position and in a closed state/position, wherein the first housing part and the second housing part are configured in the open state to receive a first dialyzer element without housing, in particular as described above, wherein the first housing part and the second housing part are lockable to each other in the closed state, and wherein the housing is configured and prepared to be connected to a dialysis fluid circuit/dialysis fluid-carrying/dialysate-carrying tubes.

The first housing part is preferably formed as a first half-shell. The second housing part is preferably formed as a second half-shell. Thus, the first housing part and the second housing part preferably form a (hollow) cylindrical housing in their closed state.

The housing is preferably configured to be transparent, i.e. made of a transparent material. Alternatively or additionally, the housing may be formed from a disinfectant-stable material, as it should be able to withstand several disinfection cycles. In particular, polymers such as thermoplastic polyester, polyethylene terephthalate (PET), polybutylene terephthalate (PBT), polymethyl methacrylate (PMMA), styrene-acrylonitrile copolymers (SAN), blends with polycarbonate, etc. can be used as materials for the housing. Non-transparent materials are also conceivable as long as they are basically non-toxic. In principle, metals, for example, may also be considered as housing materials.

In an advantageous manner, the second dialyzer element with housing is configured to be firmly and permanently attached to a blood treatment device, in particular a dialysis machine. Thus, the second dialyzer element with housing may remain permanently on the blood treatment device and does not have to be mounted before a blood treatment therapy and removed after the blood treatment therapy.

Preferably, the housing/second dialyzer element with housing comprises a third terminal and a fourth terminal, wherein the third terminal and the fourth terminal are prepared, configured or provided to be connected to a dialysis fluid circuit/dialysate circuit. Particularly preferably, the second dialyzer element with housing has exactly two terminals, i.e. the third terminal and the fourth terminal, so that the second dialyzer element with housing is prepared or configured to be connected to the dialysis fluid circuit/dialysate circuit only/exclusively via the third terminal and the fourth terminal. In other words, the second dialyzer element with housing preferably has no blood connections, i.e. no terminals which are prepared, configured or provided to be connected to an extracorporeal blood circuit. The second dialyzer element with housing is thus preferably formed as a dialysis fluid-carrying/dialysate-carrying/dialysate-contacting part of a dialyzer.

In an advantageous manner, the two terminals, i.e. the third terminal and the fourth terminal, of the second dialyzer element with housing are provided on one of the two housing parts. That is, preferably, both the third terminal and the fourth terminal are provided/arranged on the first housing part, or both the third terminal and the fourth terminal are provided/arranged on the second housing part.

Preferably, the second dialyzer element with housing is firmly and permanently attached to the blood treatment device (to a machine front thereof) at least via the dialysate terminals/dialysis fluid terminals, i.e. via the third terminal and the fourth terminal. In other words, the third terminal and the fourth terminal are thus preferably permanently connected or linked to the dialysis fluid circuit/dialysate circuit. This has the advantage that the dialysate terminals/dialysis fluid terminals do not have to be linked before a blood treatment therapy.

According to a preferred configuration example, the second dialyzer element with housing comprises a hinge about which the first housing part and/or the second housing part is/are pivotable in order to move the first housing part and the second housing part into the open state/position and into the closed state/position. Preferably, one of the housing parts is pivotable, while the other housing part is fixedly attached to the blood treatment device. For example, the first housing part is fixedly attached to the blood treatment device, while the second housing part is pivotable about the hinge. Alternatively, the second housing part is fixedly attached to the blood treatment device, while the first housing part is pivotable about the hinge.

Furthermore, the second dialyzer element with housing preferably comprises at least one closure element/closure device, for example a closure latch, which is arranged on the first housing part and/or on the second housing part and via which the first housing part and the second housing part are lockable to each other. The two housing parts may, for example, be closed or locked to each other using a tension lock, a toggle lock, etc. In principle, other closures are also conceivable as long as they are easy and safe to operate.

Preferably, the at least one closure element is arranged on the housing part on which the dialysate terminals/dialysis fluid terminals are also arranged. However, it may also be arranged on the other housing part. In addition, a plurality of closure elements, for example two, three, four, five, etc., may also be provided. The plurality of closure elements may be arranged on one and the same housing part, but may also be arranged on both housing parts, for example such that at least one closure element is arranged on the first housing part and that at least one closure element is arranged on the second housing part.

The closure/closure element is particularly preferably locked by the blood treatment device/dialysis machine so that it cannot be opened accidentally during blood treatment therapy.

An advantageous configuration examples is thus characterized by the fact that the second dialyzer element with housing can be opened and closed via the hinge and can be closed or locked via the closure element/closure device. Reusability of the second dialyzer element with housing is thus preferably made possible via the hinge and the closure element/closure device.

Preferably, the second dialyzer element with housing comprises a third sealing element or a third sealing surface at or near a first axial end of the housing and a fourth sealing element or a fourth sealing surface at or near a second axial end of the housing.

In other words, according to the disclosure, two preferred embodiments for the second dialyzer element with housing are conceivable with regard to the sealing elements or sealing surfaces. According to a first embodiment, the housing may have at least two sealing elements, which are formed, for example, in particular as injection-molded or glued-on sealing rings, which are configured or provided to interact in a sealing manner with sealing surfaces provided on a separate first dialyzer element without housing. Accordingly, the housing may have a third sealing element at/near a first axial end thereof and a fourth sealing element at/near a second axial end thereof. Alternatively, i.e. according to a second embodiment, the housing may have at least two sealing surfaces which are configured or provided to interact in a sealing manner with sealing elements provided on a separate first dialyzer element without housing. In particular, the sealing surfaces may be realized by smooth, flat, inner (cylindrical) lateral surfaces of the housing. In other words, the housing may alternatively have a third sealing surface at/near a first axial end thereof and a fourth sealing surface at/near a second axial end thereof.

By sealing the second dialyzer element with housing to the first dialyzer element without housing, which is preferably inlaid/inserted therein, via interacting sealing elements and sealing surfaces at/near the axial ends of the dialyzer formed by the two dialyzer elements, it can be achieved that dialysis fluid enters the dialyzer via a terminal of the second dialyzer element with housing (for example the third terminal) during a blood treatment therapy, then flows through the liquid-permeable sheathing of the first dialyzer element without housing in order to purify the blood flowing through the hollow fibers, and is prevented from flowing out of the dialyzer at the axial ends of the dialyzer, so that the spent dialysis fluid or dialysate can only flow out of the dialyzer via a terminal of the second dialyzer element with housing (for example the fourth terminal).

With regard to the sealing elements, it is therefore preferred that either the first dialyzer element without housing has two sealing elements or the second dialyzer element with housing has two sealing elements. In other words, two sealing elements may be fitted radially on the inside at both ends of the housing/of the second dialyzer element with housing. Alternatively, two sealing elements may also be attached radially on the outside to the end caps of the first dialyzer element without housing (one sealing element per end cap). The sealing element according to the present disclosure preferably consists of an elastic material. Silicones are particularly suitable due to their excellent biocompatibility. However, other materials such as fluoroplastics, polyaryletherketones, polyamides, polyacetates and polyethylenes are also possible.

The second dialyzer element with housing/the housing thereof may have an opening which is air-permeable and liquid-impermeable/fluid-tight. This opening is particularly preferably located on an upper portion of the housing during an ongoing blood treatment therapy. This opening advantageously enables complete venting and removal of bubbles that arise during blood treatment therapy due to outgassing of bicarbonate. The opening may be provided on the first housing part or on the second housing part.

Furthermore, the second dialyzer element with housing may have at least two sealing plugs/filler plugs, which can be inlaid into the housing in order to seal the housing at its axial ends in a liquid-tight manner before cleaning/disinfecting the dialysis fluid circuit together with the second dialyzer element with housing. The sealing plugs/filler plugs may be permanently connected to the housing and may be inlaid/inserted into the housing or may be removed again as required. Alternatively, the sealing plugs/filler plugs may also be permanently fixed to a machine front of a blood treatment device, in particular in the vicinity of the second dialyzer element with housing, which is permanently attached to the machine front. However, it is also conceivable to form the sealing plugs/filler plugs separately from both the housing and the machine front of the blood treatment device, in particular as separate individual components.

The second dialyzer element with housing may comprise a housing insert separate from the housing/two housing parts, which may also be referred to as the third housing part.

Such a housing insert may in particular be formed in such a way that it obstructs a dialysis fluid flow/dialysate flow, in particular in an axial direction of the housing in a potential clearance between the housing of the second dialyzer element with housing and the sheathing of the first dialyzer element without housing. As a result, a higher packing density, a higher inner or internal filtration and thus a higher dialysis performance/clearance can be achieved in an advantageous manner. This also makes it possible that a smaller fiber surface of the hollow fiber bundle suffices.

It may be advantageous if the housing insert is made of an elastic material and is then pressed into the hard shape of the first housing part and the second housing part. The dialysis performance/clearance may be further improved by the elastic material of the housing insert and the thus improved compression of the capillaries/of the hollow fiber bundle. Alternatively, however, the housing insert may also be made of a rigid or solid material, such as a thermoplastic.

According to a preferred configuration example, the housing insert may have an elevation/barrier in an (axial) middle portion in order to achieve a particularly strong compression in the middle of the dialyzer in particular. This further increases the flow resistance for the dialysis fluid/dialysate, resulting in increased convective exchange and increased clearance of large molecules.

The second dialyzer element with housing may have at least one sensor installed on the housing. For example, a sensor configured to measure diagnostically relevant markers may be attached to the housing. In particular, at least one pressure sensor may be installed inside the housing. For example, if a housing insert with an elevation/barrier is provided in the housing, two pressure sensors, one before the barrier and one after the barrier, may be installed in the housing. The pressure values measured by these pressure sensors can be used to draw conclusions about the convective exchange within the dialyzer.

In principle, the second dialyzer element with housing may also be connected to a dialysis fluid-carrying/dialysate-carrying cassette permanently installed in a machine housing of a blood treatment device, which in turn has terminals for incoming dialysis fluid and outgoing/discharged dialysis fluid/dialysate. According to this embodiment, the second dialyzer element with housing is preferably connected to the dialysis fluid circuit/dialysate circuit via this cassette.

Furthermore, the present disclosure relates to a dialyzer comprising the first dialyzer element/part without housing as described above and the second dialyzer element/part with housing as described above. Preferably, the first dialyzer element without housing and the second dialyzer element with housing are separate/discrete components and are each constituents of the multi-part dialyzer of the present disclosure.

Preferably, the first dialyzer element without housing is inlaid/inserted into the second dialyzer element with housing. In particular, the first dialyzer element without housing is only inlaid into the second dialyzer element with housing immediately or shortly before a blood treatment therapy.

In the closed state of the second dialyzer element with housing, the second dialyzer element with housing preferably completely surrounds the first dialyzer element without housing.

The second dialyzer element with housing and the first dialyzer element without housing are preferably sealed to each other, in particular via corresponding sealing elements and sealing surfaces. The sealing preferably takes place at/near (the two) axial ends of the dialyzer.

Sealing may take place via sealing elements (radially outside) on the end caps of the first dialyzer element without housing, which in the closed state of the two housing parts of the second dialyzer element with housing seal against an inner lateral surface/circumferential surface/sealing surface of the second dialyzer element with housing.

Alternatively, sealing may also take place via sealing elements (radially inside) on the housing/the two housing parts, which in the closed state of the two housing parts of the second dialyzer element with housing seal against an outer lateral surface/circumferential surface/sealing surface of the end caps of the first dialyzer element without housing.

In an advantageous manner, the first dialyzer element/part without housing is configured/prepared to be connected to an extracorporeal circuit, and the second dialyzer element/part with housing is configured/prepared to be connected to a dialysis fluid circuit. In this context, the first dialyzer element/part without housing preferably has a first terminal (at the first axial end) and a second terminal (at the second axial end), and the second dialyzer element/part with housing preferably has a third terminal and a fourth terminal (at one of the two housing parts).

Furthermore, the present disclosure relates to a blood treatment device, in particular a dialysis machine, comprising a machine front and the second dialyzer element with housing as described above, wherein the second dialyzer element with housing is fixedly attached or installed to the machine front. The fixed attachment or installation of the second dialyzer element with housing to the machine front is preferably carried out via the dialysis fluid terminals/dialysate terminals.

Particularly preferred are means provided on the blood treatment device/machine front which enable the closure device/closure element of the second dialyzer element with housing to be locked, so that accidental opening of the housing during an ongoing treatment can be ruled out.

In principle, according to the disclosure, it may also be provided that a plurality of second dialyzer elements with housings are permanently attached or installed on the blood treatment device. For example, several second dialyzer elements with housings may be arranged in a turret configuration and can be rotated into the foreground by a user in order to insert a first dialyzer element without housing. In particular, this may shorten the preparation time from patient to patient, for example if several first dialyzer elements without housings are inserted into corresponding second dialyzer elements with housings in advance. In addition, different dialyzer sizes may also be realized with this embodiment.

Furthermore, the disclosure relates to a method for reprocessing a second dialyzer element with housing, in particular as described above, after a blood treatment therapy, comprising the steps of: a) removing a first dialyzer element without housing, in particular as described above, from the second dialyzer element with housing; and b) cleaning and/or disinfecting the second dialyzer element with housing.

Preferably, the method further comprises the step of: c) inserting a first sealing plug/filler plug and a second sealing plug/filler plug into the second dialyzer element with housing; wherein step c) is performed between step a) and step b).

In particular, according to the present disclosure, the first dialyzer element without housing is inserted or inlaid into the second dialyzer element with housing, which is preferably attached to the blood treatment device. By closing or locking the second dialyzer element with housing, sealing via corresponding sealing elements and sealing surfaces of the two dialyzer elements to each other at axial ends of the dialyzer formed by the two dialyzer elements is preferably carried out automatically. Blood treatment therapy is preferably carried out in this condition. During blood treatment therapy, blood flows through the first dialyzer element without housing, in particular through the end caps and the hollow fiber bundle. Dialysis fluid flows into the housing via one of the terminals of the second dialyzer element with housing, for example via the third terminal, and through the sheathing of the first dialyzer element without housing into the clearance between the individual hollow fibers of the hollow fiber bundle, so that the blood flowing through the hollow fibers can be purified. Spent dialysis fluid/dialysate flows out of the housing via one of the terminals of the second dialyzer element with housing, for example via the fourth terminal. After the blood treatment therapy, the dialysate still inside the housing is preferably sucked out of the housing as far as possible.

This ‘sucking of the dialysis fluid/dialysate from the housing/the second dialyzer element/part with housing’ preferably represents the first step of the method according to the disclosure for reprocessing a second dialyzer element with housing.

In a second step ‘opening of the second dialyzer element with housing’, the closure of the second dialyzer element with housing is advantageously opened and one of the housing parts is pivoted around the hinge into the open state of the housing parts.

Subsequent to this second step, step a) defined above is carried out in a third step and step c) defined above is carried out in a fourth step. In a fifth step ‘closing of the second dialyzer element with housing’, one of the housing parts is advantageously pivoted about the hinge into the closed state of the housing parts and the closure of the second dialyzer element with housing is closed again. Finally, in a sixth step, step b) defined above follows. In the context of step b), a disinfection cycle/cleaning cycle is started on the machine side and the dialysis fluid circuit/dialysate circuit is disinfected together with the second dialyzer element with housing.

In summary, the present disclosure relates to:
a first dialyzer element without housing comprising a hollow fiber bundle, a liquid-permeable sheathing circumferentially wrapped around the hollow fiber bundle, cast elements at the axial ends of the hollow fiber bundle and end caps attached to the cast elements for connection to an extracorporeal blood circuit;
a lockable, openable and reusable second dialyzer element with a housing;
a dialyzer comprising the first dialyzer element without housing and the second dialyzer element with housing;
a blood treatment device comprising a machine front and the second dialyzer element with housing fixedly attached to the machine front; and
a method for reprocessing the second dialyzer element with housing after a blood treatment therapy.

The present disclosure provides the following overall advantages: by providing a reusable housing (the second dialyzer element with housing), plastic waste can be reduced since only the components of the dialyzer (the first dialyzer part without housing) that are actually contaminated with blood are (will be) disposed of. The housing/the second dialyzer part with housing can remain firmly attached to the dialysis machine/the blood treatment device, so that no dialysis fluid terminals/dialysate terminals need to be connected before a blood treatment therapy. A higher internal filtration can be achieved by providing or preparing inserts in the housing which impede the flow of dialysis fluid/dialysate. Compared to a classic dialyzer, a higher packing density can be achieved, which is associated with a higher clearance (reduction of the urea content of the blood). It is therefore also possible that a smaller fiber surface of the hollow fiber bundle is sufficient. In addition, it is possible to measure diagnostically relevant markers in or on the housing and thus within the dialyzer using sensors (permanently installed on the housing).

### BRIEF DESCRIPTION OF THE DRAWINGS

The disclosure is further explained below with the aid of Figures.

FIG. 1 shows a longitudinal sectional view of a first dialyzer element without housing according to a first embodiment of the present disclosure;

FIG. 2 shows a cross-sectional view of the first dialyzer element without housing at a line A-A in FIG. 1

FIG. 3 shows a perspective view of a second dialyzer element with housing according to the first embodiment of the present disclosure;

FIG. 4 shows a sectional view of a dialyzer in which the first dialyzer element without housing is inserted into the second dialyzer element with housing, according to a second embodiment of the present disclosure;

FIG. 5 shows a perspective view of a housing insert according to the present disclosure;

FIG. 6 shows a schematic view of a cassette system with a blood-carrying, exchangeable cassette and a dialysis fluid-carrying/dialysate-carrying, permanently installed cassette;

FIG. 7 shows a flowchart of a method according to the disclosure for reprocessing a second dialyzer element with housing after a blood treatment therapy; and

FIG. 8 shows a sectional view of the second dialyzer element with housing during the reprocessing process.

### DETAILED DESCRIPTION

The Figures are merely schematic in nature and are intended solely to improve understanding of the present disclosure. Identical elements are provided with the same reference signs. Features of the individual embodiments/examples can be interchanged.

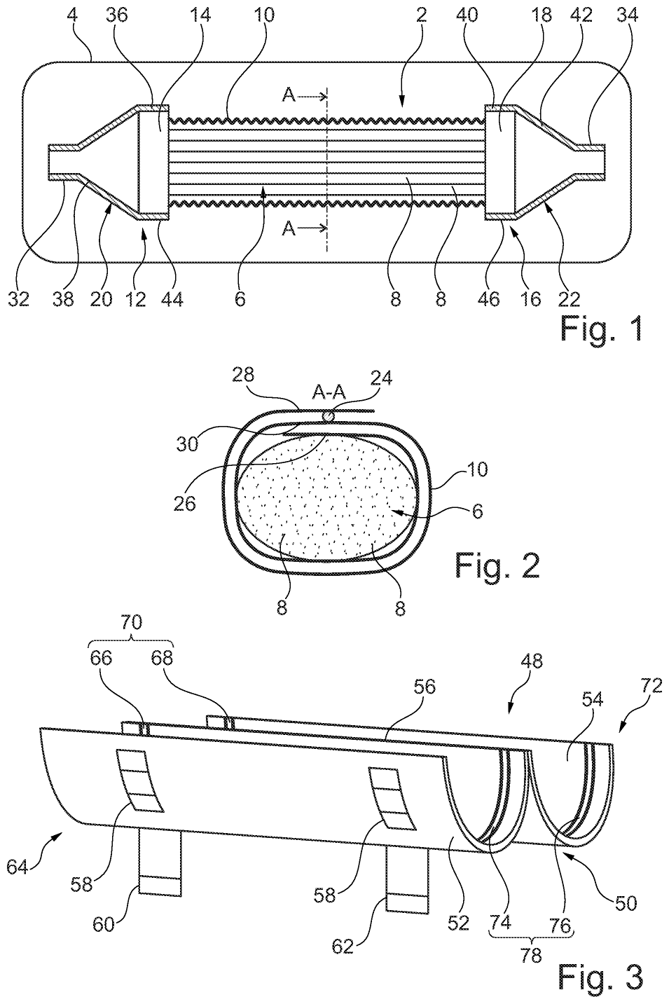

FIG. 1 shows a first dialyzer element or respectively dialyzer part 2 without housing, which is packaged in sterile packaging 4. The first dialyzer element 2 without housing has a substantially rotationally symmetrical, cylindrical shape. The first dialyzer element without housing has a hollow fiber bundle 6, which consists of a plurality of hollow fibers 8. The hollow fiber bundle 6 is circumferentially wrapped by a sheathing/sheathing film 10 and thus packaged. A first cast element/a first cast cap 14 is provided at a first axial end 12 of the hollow fiber bundle 6. A second cast element/a second cast cap 18 is provided at a second axial end 16 of the hollow fiber bundle 6. The first cast element 14 and the second cast element 18 are preferably produced by casting or pouring the hollow fiber bundle 6 together with the sheathing 10 at its axial ends 12, 16 using a casting compound made of polyurethane or silicone and then cutting it. A first end cap/blood cap 20 is attached to the first cast element 14, in particular by adhesive bonding. A second end cap/blood cap 22 is attached to the second cast element 18, in particular by adhesive bonding.

The sheathing 10 is formed as a textile surface structure, preferably as a coarse-meshed fabric, nonwoven or net. The sheathing 10 bundles the hollow fibers 8 into the hollow fiber bundle 6 and holds them together on the circumferential side. The sheathing 10 is liquid-permeable and, in particular, allows dialysis fluid or dialysate to flow through it. It also allows the casting compound to flow through it. As can be seen from FIG. 2, the sheathing 10 is preferably wrapped more than once around the hollow fiber bundle 6. A secure closure of the sheathing is achieved by a welding 24. In FIG. 2, an inner layer 26 and an outer layer 28 of the sheathing 10 overlap. In addition, an intermediate layer 30 of the sheathing 10 is provided, so that sectionally three layers 26, 28, 30 of the sheathing 10 lie on top of each other. The welding 24 is provided between the outer layer 28 and the intermediate layer 30. The inner layer 26 serves as a protective layer for the hollow fibers 8 of the hollow fiber bundle 6.

The first end cap 20 has a first terminal 32. The second end cap 22 has a second terminal 34. The first terminal 32 and the second terminal 34 serve to connect the first dialyzer element 2 without housing to blood-carrying tubes of an extracorporeal blood circuit. During a blood treatment therapy, extracorporeal blood of a patient flows into the first dialyzer element 2 without housing via the first terminal 32 of the first end cap 20, flows through the hollow fibers 8, and leaves the first dialyzer element 2 without housing via the second terminal 34 of the second end cap 22. The first end cap 20 has a first hollow-cylindrical portion 36 and a first tapering portion 38 extending towards the first terminal 32. Thus, a diameter of the first hollow-cylindrical portion 36 is greater than a diameter of the first terminal 32. The second end cap 32 has a second hollow-cylindrical portion 40 and a second tapering portion 42 extending towards the second terminal 34. Thus, a diameter of the second hollow-cylindrical portion 40 is larger than a diameter of the second terminal 34. The first cast element 14 is inserted into the first hollow-cylindrical portion 36 and fixed therein, in particular by adhesive bonding. The second cast element 18 is inserted into the second hollow-cylindrical portion 40 and fastened therein, in particular by adhesive bonding.

The first end cap 20 has a first sealing surface 44 on the outer circumference of the first hollow-cylindrical portion 36. The second end cap 22 has a second sealing surface 46 on the outer circumference of the second hollow-cylindrical portion 40. The first sealing surface 44 and the second sealing surface 46 are characterized by the fact that they are smooth, flat surfaces with an invariable outer diameter, i.e. smooth, flat cylindrical lateral surfaces, so that they are particularly suitable for interacting in a sealing manner with a sealing element formed as a sealing ring.

FIG. 3 shows a second dialyzer element or respectively dialyzer part 48 with housing. The second dialyzer element 48 with housing is formed in the present case from a closable, openable and thus reusable housing 50. The housing 50 has a first housing part 52 formed as a half-shell and a second housing part 54 formed as a half-shell. The two housing parts 52, 54 are pivotable relative to each other about a hinge 56. FIG. 3 shows an open state of the two housing parts 52, 54. In the state shown in FIG. 3, the first dialyzer element 2 without housing shown in FIG. 1 can be inserted into the second dialyzer element 48 with housing. If, for example, the second housing part 54 is pivoted about the hinge 56 from the state shown in FIG. 3, the two housing parts 52, 54 can be brought into a closed state in which they form a hollow-cylindrical housing 50. In the closed state, the two housing parts 52, 54 can be locked via two closure elements 58 attached to the first housing part 52. Furthermore, two terminals, namely a third terminal 60 and a fourth terminal 62, are arranged on the first housing part 52, which extend away from the first housing part 52 parallel to each other in the radial direction of the first housing part 52.

At or near a first axial end 64 of the housing 50, the first housing part 52 has a first sealing half ring 66 and the second housing part 54 has a second sealing half ring 68. In the closed state of the two housing parts 52, 54, the first sealing half ring 66 and the second sealing half ring 68 together form a circumferential, uninterrupted sealing ring 70. At or near a second axial end 72 of the housing 50, the first housing part 52 has a third sealing half ring 74 and the second housing part 54 has a fourth sealing half ring 76. In the closed state of the two housing parts 52, 54, the third sealing half ring 74 and the fourth sealing half ring 76 together form a circumferential, uninterrupted second sealing ring 78.

When the first dialyzer element 2 without housing shown in FIG. 1 is inlaid in the second dialyzer element 48 with housing shown in FIG. 3, and the two housing parts 52, 54 of the housing 50 of the second dialyzer element 48 with housing are in the closed state and locked via the closure elements 58, the first sealing ring 70 interacts in a sealing manner with the first sealing surface 44 and the second sealing ring 78 interacts in a sealing manner with the second sealing surface 46.

The first dialyzer element 2 without housing shown in FIG. 1 and the second dialyzer element 48 shown in FIG. 3 together form a dialyzer 80 according to a first embodiment, according to which sealing elements, in this case the sealing rings 70, 78, are provided on the second dialyzer element 48 with housing, and sealing surfaces, in this case the sealing surfaces 44, 46, are provided on the first dialyzer element 2 without housing.

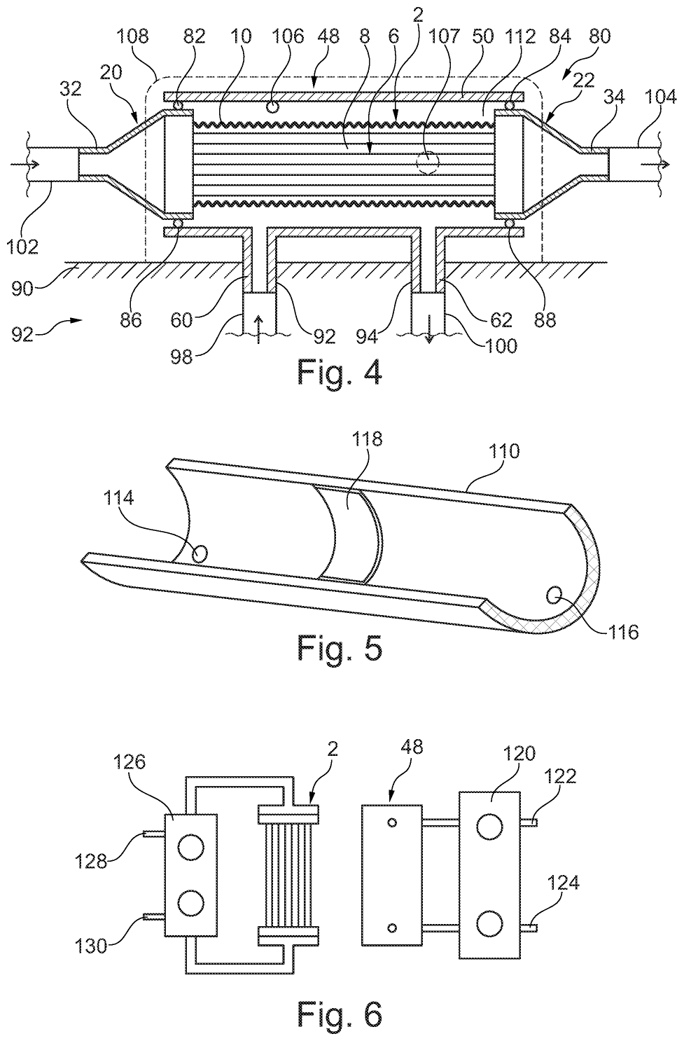

FIG. 4 shows a dialyzer 80 according to a second embodiment. The dialyzer 80 has the first dialyzer element 2 without housing and the second dialyzer element 48 with housing. The preceding descriptions of the first dialyzer element 2 without housing and the second dialyzer element 48 with housing are substantially mutatis mutandis applicable to the embodiment shown in FIG. 4 and are therefore not repeated. However, according to the second embodiment shown in FIG. 4, the first dialyzer element 2 without housing contains sealing elements which are formed here as sealing rings 82, 84, wherein the third sealing ring 82 is molded or glued onto the first end cap 20 and wherein the fourth sealing ring 84 is molded or glued onto the second end cap 22. According to the second embodiment shown in FIG. 4, sealing surfaces 86, 88 are provided on the second dialyzer element 48 with housing, wherein the sealing surfaces interact with the sealing rings 82, 84 in the assembled condition of the dialyzer 80.

In FIG. 4, the second dialyzer element 48 with housing is firmly attached to a machine front 90 of a dialysis machine 92 by plugging the terminals 60, 62 into corresponding receiving recesses 92, 94 provided on the machine front 90. The third terminal 60 is thus connected to a dialysis fluid inlet tube/dialysis fluid inlet line 98. The fourth terminal 62 is thereby connected to a dialysate outlet tube/dialysate outlet line 100. As can also be seen from FIG. 4, the first terminal 32 of the first end cap 20 of the first dialyzer element 2 without housing is connected to a blood inlet tube/blood inlet line 102, and the second terminal 34 of the second end cap 22 of the first dialyzer element 2 without housing is connected to a blood outlet tube/blood outlet line 104.

During blood treatment therapy, a patient's blood flows from the blood inlet tube 102 into the first end cap 20, flows through the hollow fibers 8 of the hollow fiber bundle 6 and flows out of the first dialyzer element 2 without housing via the second end cap 22 into the blood outlet tube 104. Dialysis fluid flows from the dialysis fluid inlet tube 98 into the third terminal 60 and flows through the liquid-permeable sheathing 10 of the first dialyzer element 2 without housing into the clearance between the hollow fibers 8 of the hollow fiber bundle 6, so that the blood flowing through the hollow fibers 8 can be purified. Finally, used dialysis fluid or dialysate flows out of the housing 50 of the second dialyzer element 48 with housing via the fourth terminal 62 into the dialysate outlet tube 100.

A pressure sensor 106 installed in the housing 50 can measure a pressure within the housing 50 during the blood treatment therapy. The housing 50, i.e. one of the two housing parts 52, 54, also has an air-permeable and liquid-impermeable opening 107, indicated by a dashed line in FIG. 4, which enables venting and removal of bubbles that arise during the blood treatment therapy due to outgassing of bicarbonate. This opening 107 is located at the top in the vertical direction of the dialysis machine 92/of the housing 50 (i.e. in a direction perpendicular to the sectional plane shown in FIG. 4). In addition, a pivotable and lockable cover 108, indicated by a dashed line in FIG. 4, is also provided on the machine front 90 of the dialysis machine 92, which covers the closure elements 58 of the second dialyzer element 48 with housing during blood treatment therapy so that they cannot be opened inadvertently. The closure elements 58 are thus inaccessible to a user, i.e. locked, by the lockable cover 108 of the dialysis machine 92.

A housing insert 110 shown in FIG. 5 can be inlaid into the housing 50 of the second dialyzer element 48 with housing, which can fill a clearance 112 between the housing 50 and the sheathing 10. The housing insert 110 may be half-shell-like as shown in FIG. 5, but can alternatively also be cylindrical. It is also conceivable to insert two half-shell-like housing inserts 110 into the housing 50. The housing insert 110 may be made of an elastic or rigid plastic material and has an inlet opening 114 and an outlet opening 116. The housing insert 110 is preferably arranged in the housing 50 such that the inlet opening 114 is provided adjacent to and aligned with the third terminal 60 and that the outlet opening 116 is provided adjacent to and aligned with the fourth terminal 62 so that the housing insert 110 does not obstruct the dialysis fluid inflow and the dialysate outflow. The inlet opening 114 and the outlet opening 116 are thus preferably arranged near the axial ends of the housing insert 110. In a central portion of the housing insert 110, this has an elevation or projection or barrier 118.

As can be seen from FIG. 6, the second dialyzer element 48 with housing may also be connected to a dialysis fluid-carrying/dialysate-carrying cassette 120 permanently installed on the dialysis machine 92, which in turn has terminals 122, 124 for inflowing dialysis fluid and dialysate, and the first dialyzer element 2 without housing can also be connected to an exchangeable blood-carrying cassette 126, which in turn has terminals 128, 130 for inflowing and outflowing blood.

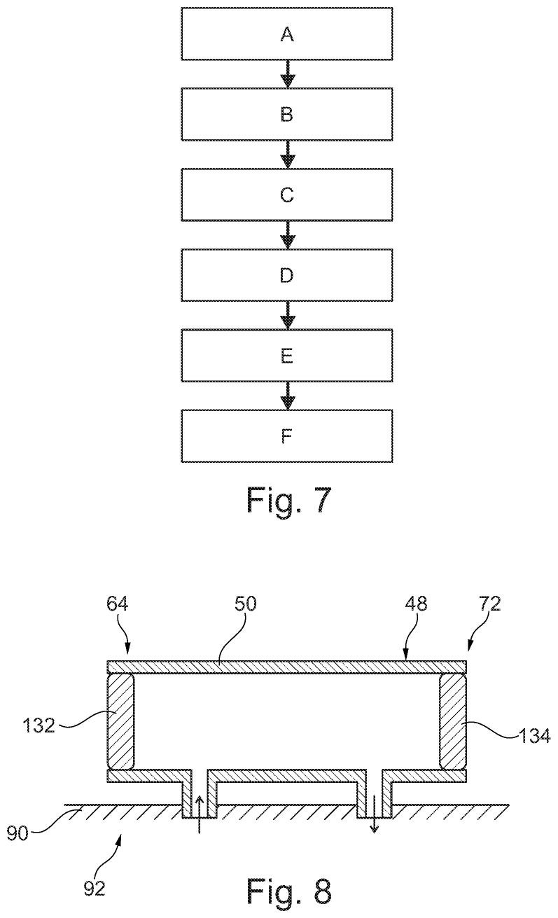

The flowchart in FIG. 7 shows in more detail the method according to the disclosure for reprocessing the second dialyzer element 48 with housing after a blood treatment therapy. The process sequence is as follows:
A: sucking the dialysis fluid or dialysate from the second dialyzer element 48 with housing.
B: opening of the second dialyzer element 48 with housing.
C: removing the first dialyzer element 2 without housing from the second dialyzer element 48 with housing.
D: inserting a first sealing plug/filler plug 132 and a second sealing plug/filler plug 134 into the second dialyzer element 48 with housing.
E: closing the second dialyzer element 48 with housing.
Q: cleaning and/or disinfecting the second dialyzer element 48 with housing.

FIG. 8 shows the second dialyzer element 48 with housing with inlaid sealing plugs/filler plugs 132, 134. The sealing plugs 132, 134 interact here in a sealing manner with the housing 50, so that the second dialyzer element 48 with housing is sealed at its axial ends 64, 72. In this condition, cleaning or disinfection cycle can be started on the machine side in order to clean or disinfect and thus reprocess the second dialyzer element 48 with housing.

1. A first dialyzer element without housing comprising:
a hollow fiber bundle comprising a first axial end and a second axial end;
a liquid-permeable sheathing circumferentially wrapped around the hollow fiber bundle;
a first cast element provided at the first axial end of the hollow fiber bundle and a second cast element provided at the second axial end of the hollow fiber bundle; and
a first end cap attached to the first cast element and a second end cap attached to the second cast element, wherein
the first dialyzer element without housing is connectible to an extracorporeal blood circuit.

2. The first dialyzer element without housing according to claim 1, wherein the liquid-permeable sheathing is formed as a textile surface structure.

3. The first dialyzer element without housing according to claim 1, which is packaged sterile in a sterile packaging.

4. The first dialyzer element without housing according to claim 1, wherein the first end cap has a first sealing surface or a first sealing element on an outer circumference of the first end cap, and the second end cap has a second sealing surface or a second sealing element on an outer circumference of the second end cap.

5. A second dialyzer element with housing, which is formed as, or comprises, a closable, openable and reusable housing, the second dialyzer comprising:
a first housing part and a second housing part which are configured to be brought in an open state and in a closed state, wherein:
the first housing part and the second housing part are configured in the open state to receive a first dialyzer element without housing;
the first housing part and the second housing part are lockable to each other in the closed state; and
the housing is connectable to a dialysis fluid circuit.

6. The second dialyzer element with housing according to claim 5, wherein the second dialyzer element with housing is configured to be firmly and permanently attached to a blood treatment device.

7. The second dialyzer element with housing according to claim 5, further comprising a hinge about which the first housing part and/or the second housing part is pivotable in order to move the first housing part and the second housing part into the open state and into the closed state.

8. The second dialyzer element with housing according to claim 5, further comprising at least one closure element which is arranged on the first housing part and/or on the second housing part and via which the first housing part and the second housing part are lockable to each other.

9. The second dialyzer element with housing according to claim 5, further comprising a third sealing element or a third sealing surface at or near a first axial end of the housing and a fourth sealing element or a fourth sealing surface at or near a second axial end of the housing.

10. The second dialyzer element with housing according to claim 5, further comprising a housing insert separate from the housing and/or a sensor installed on the housing.

11. A dialyzer comprising:
the first dialyzer element without housing according to claim 1; and
a second dialyzer element with housing which is formed as, or comprises, a closable, openable and reusable housing, the second dialyzer element comprising:
a first housing part and a second housing part which are configured to be brought in an open state and in a closed state, wherein:
the first housing part and the second housing part are configured in the open state to receive the first dialyzer element without housing;
the first housing part and the second housing part are lockable to each other in the closed state; and
the housing is connectable to a dialysis fluid circuit.

12. The dialyzer according to claim 11, wherein
the first dialyzer element without housing is inlaid into the second dialyzer element with housing,
in the closed state of the second dialyzer element with housing, the second dialyzer element with housing completely surrounds the first dialyzer element without housing, and
the second dialyzer element with housing and the first dialyzer element without housing are sealed to each other via corresponding sealing elements and sealing surfaces.

13. A blood treatment device comprising:
the second dialyzer element with housing according to claim 5; and
a machine front,
wherein the second dialyzer element with housing is firmly attached or installed on the machine front.

14. A method for reprocessing a second dialyzer element with housing after a blood treatment therapy, the method comprising the steps of:
a) removing a first dialyzer element without housing from the second dialyzer element with housing; and
b) cleaning and/or disinfecting the second dialyzer element with housing.

15. The method according to claim 14, further comprising the step of:
c) inserting a first sealing plug and a second sealing plug into the second dialyzer element with housing; wherein
step c) is performed between step a) and step b).
